(19) United States
(12) Patent Application Publication
(10) Pub. No.: US 2017/0242032 A1
(43) Pub. Date: Aug. 24, 2017

(54) METHOD FOR ANALYSING AGGREGATES IN ANTIBODY SAMPLES

(71) Applicant: GENOVIS AB, Lund (SE)

(72) Inventors: Sarah FREDRIKSSON, Dalby (SE); Magdalena Widgren Sandberg, Lund (SE); Fredrik Olsson, Kavlinge (SE)

(21) Appl. No.: 15/512,505

(22) PCT Filed: Sep. 22, 2015
(86) PCT No.: PCT/EP2015/071771

§ 371 Date: Mar. 17, 2017

(30) Foreign Application Priority Data
Sep. 24, 2014 (GB) 1416849.6

(51) Int. Cl. G01N 33/68

(52) U.S. Cl. CPC G01N 33/6854; G01N 2333/952

(57) ABSTRACT

The invention provides methods for analysing a sample of immunoglobulins to determine the amount of IgG aggregates present in the sample.

### FIELD OF THE INVENTION

The present invention relates to methods for analysing a sample of immunoglobulins.

### BACKGROUND OF THE INVENTION

The characterisation of antibodies, such as structural characterisation and physiochemical analysis, is required by developers and producers of antibody based therapeutics. Antibodies produced for therapeutic use are subject to quality control to ensure that the antibodies as produced have necessary binding characteristics and stability. One of the key qualities to be assessed is the extent if any of any aggregation of antibodies for therapeutic use. Such aggregation is detrimental to the overall quality and effectiveness of the antibodies. Previously, size exclusion chromatography (SEC) has been used to assess the amount of aggregates in an antibody preparation. This is a low throughput method. Improved methods for the quantification of aggregates in an antibody preparation are required.

### SUMMARY OF THE INVENTION

A cysteine protease enzyme from Streptococcus pyogenes, immunoglobulin G-degrading enzyme of S. pyogenes (IdeS) has been reported to have the activity of cleaving IgG antibodies to produce Fc and Fab fragments. The present inventors have identified that aggregated antibodies are able to withstand IdeS digestion, and that there is a direct relationship between the amount of aggregated IgG in a sample and the amount of IgG amenable to digestion by IdeS. Accordingly, the present invention is directed to the use of an IdeS polypeptide to quantify the amount of aggregated antibody present in a sample. The use of IdeS provides the opportunity to analyse samples and quantify the amount of aggregates therein using high throughput methods.

In accordance with the present invention, there is a method for quantifying the amount of IgG antibody aggregate in a sample of IgG immunoglobulin molecules comprising

(a) contacting the sample with a IdeS polypeptide under conditions which allow cleavage of an unaggregated IgG antibody,

(b) quantifying a cleavage product produced in step (a), and

(c) using the result of step (b) to determine the amount of antibody aggregation in the sample.

### BRIEF DESCRIPTION OF THE FIGURES

FIG. 1 shows a SEC-HPLC chromatogram of MabThera® with 3 different aggregate concentrations after 30 minutes digestion with IdeS.

FIG. 2 shows graphs showing digestion product peak areas from SEC-HPLC chromatograms for increasing aggregate concentrations with linear regression. a) F(ab′)₂ peak area (R²=0.9989), b) Fc peak area (R²=0.9885) and c) summarized F(ab′)₂ and Fc peak areas (R²=0.9979). The data comes from three trials for enzyme digestion time 30 minutes.

FIG. 3 shows a F(ab′)₂ peak area divided with the Fc peak area from SEC-HPLC chromatogram for increasing aggregate concentrations with linear regression (R²=0.9758). The data comes from three trials for enzyme digestion time 30 minutes and one with digestion time 15 minutes.

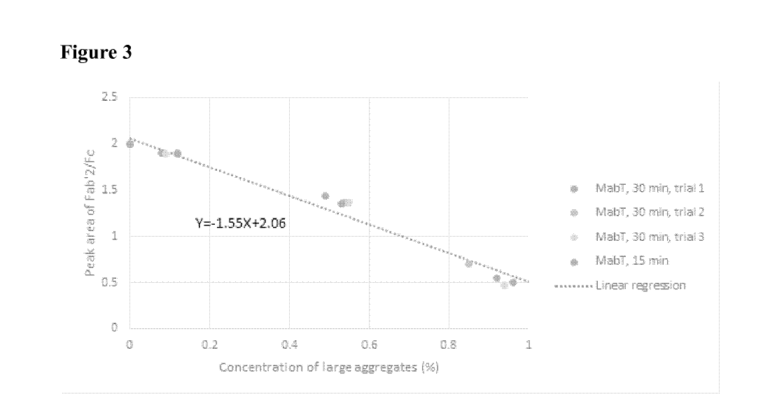

### BRIEF DESCRIPTION OF THE SEQUENCES

SEQ ID NO: 1 is an amino acid sequence encoding IdeS isolated from S. pyogenes AP1.

SEQ ID NO: 2 is an amino acid sequence encoding IdeS isolated from S. pyogenes AP1, including a putative signal sequence.

### DETAILED DESCRIPTION OF THE INVENTION

It is to be understood that different applications of the disclosed methods and products may be tailored to the specific needs in the art. It is also to be understood that the terminology used herein is for the purpose of describing particular embodiments of the invention only, and is not intended to be limiting. In addition as used in this specification and the appended claims, the singular forms “a”, “an”, and “the” include plural referents unless the content clearly dictates otherwise. Thus, for example, reference to “an immunoglobulin” includes two or more such immunoglobulins, and the like. All publications, patents and patent applications cited herein, whether supra or infra, are hereby incorporated by reference in their entirety.

The invention provides a method for analysing a sample of immunoglobulin molecules, comprising contacting the sample with an IdeS polypeptide. The sample typically contains at least one IgG molecule, and the method is typically carried out ex vivo, preferably in vitro. The IdeS polypeptide is used to cleave antibody in a sample. The amount of cleavage products is determined and is related to the amount of antibody in the preparation which is not in aggregated form, since IdeS has reduced activity for cleavage of larger aggregates. Thus, in accordance with the method, the amount of cleavage product produced is used to provide a correlation with the amount of aggregated antibody, such that the level of aggregates in the sample can be determined.

The IdeS polypeptide is an enzyme, specifically a cysteine protease enzyme, which cleaves IgG, preferably human IgG, in the hinge region of the heavy chain.

The IdeS polypeptide is preferably an IdeS polypeptide from S. pyogenes. The IdeS polypeptide may also be from another organism, such as another Streptococcus bacterium. The Streptococcus is preferably a group A Streptococcus, a group C Streptococcus or a group G Streptococcus. In particular, the IdeS polypeptide may be from a group C Streptococcus such as S. equii or S. zooepidemicus. Alternatively, the IdeS polypeptide may be from Pseudomonas putida. The IdeS polypeptide preferably comprises or consists of the amino acid sequence set forth in SEQ ID NOs: 1 or 2.

The IdeS polypeptide cleaves the hinge region of IgG between positions 249 and 250 according to the Kabat numbering system (positions 236 and 237 according to EU numbering system). An IdeS polypeptide may be obtained by any suitable means. For example, it may be isolated from any suitable organism that expresses it, such as the S. pyogenes bacterium, or it may be produced by recombinant means. IdeS polypeptides are commercially available.

For the purposes of the method of the invention, the IdeS polypeptide may be replaced with a variant or fragment thereof, provided said variant or fragment retains the functional characteristics of the original polypeptide. Specifically, the variant or fragment must retain the IgG cysteine protease activity and cleave IgG.

The cysteine protease activity of any polypeptide may be determined by means of a suitable assay. For example, a test polypeptide may be incubated with IgG at a suitable temperature, such as 37° C. The starting materials and reaction products may then be analysed by SDS-PAGE to determine whether the desired IgG cleavage product is present. The cleavage product may be subjected to N-terminal sequencing to verify that cleavage has occurred in the hinge region of IgG. The cysteine protease activity of the polypeptide can be further characterised by inhibition studies. Preferably, the activity is inhibited by the peptide derivative Z-LVG-CHN₂ and/or by iodoacetic acid both of which are protease inhibitors. However, the IdeS polypeptide (or a variant or fragment thereof) is generally not inhibited by E64.

Variants of the IdeS polypeptide may include polypeptides which have at least 80%, at least, 85%, preferably at least 90%, at least 95%, at least 98% or at least 99% identity to SEQ ID NOs: 1 or 2. The identity of variants of SEQ ID NOs: 1 or 2 can be measured over a region of at least 50, at least 100, at least 200, at least 300 or more contiguous amino acids of the sequence shown in SEQ ID NOs: 1 or 2, or more preferably over the full length of SEQ ID NOs: 1 or 2.

Amino acid identity may be calculated using any suitable algorithm. For example the PILEUP and BLAST algorithms can be used to calculate identity or line up sequences (such as identifying equivalent or corresponding sequences (typically on their default settings), for example as described in Altschul S. F. (1993) J Mol Evol 36:290-300; Altschul, S, F et al (1990) J Mol Biol 215:403-10. Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two polynucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001. Alternatively, the UWGCG Package provides the BESTFIT program which can be used to calculate identity (for example used on its default settings) (Devereux et al (1984) Nucleic Acids Research 12, 387-395).

Variants may include allelic variants and the substitution, deletion or insertion of single amino acids or groups of amino acids within the protein sequence. Variant sequences may differ by at least 1, 2, 5, 10, 20, 30, 50 or more mutations (which may be substitutions, deletions or insertions of amino acids) when compared to an original sequence. For example, from 1 to 50, 2 to 30, 3 to 20 or 5 to 10 amino acid substitutions, deletions or insertions may be made. Substitution variants preferably involve the replacement of one or more amino acids with the same number of amino acids and making conservative amino acid substitutions. For example, an amino acid may be substituted with an alternative amino acid having similar properties, for example, another basic amino acid, another acidic amino acid, another neutral amino acid, another charged amino acid, another hydrophilic amino acid, another hydrophobic amino acid, another polar amino acid, another aromatic amino acid or another aliphatic amino acid. Some properties of the 20 main amino acids which can be used to select suitable substituents are as follows:

| | | | |
|---|---|---|---|
| Ala | aliphatic, hydrophobic, | Met | hydrophobic, neutral |
| | neutral | | |
| Cys | polar, hydrophobic, | Asn | polar, hydrophilic, |
| | neutral | | neutral |
| Asp | polar, hydrophilic, | Pro | hydrophobic, neutral |
| | charged (−) | | |
| Glu | polar, hydrophilic, | Gln | polar, hydrophilic, |
| | charged (−) | | neutral |
| Phe | aromatic, hydrophobic, | Arg | polar, hydrophilic, |
| | neutral | | charged (+) |
| Gly | aliphatic, neutral | Ser | polar, hydrophilic, |
| | | | neutral |
| His | aromatic, polar, | Thr | polar, hydrophilic, |
| | hydrophilic, charged (+) | | neutral |
| Ile | aliphatic, hydrophobic, | Val | aliphatic, hydrophobic, |
| | neutral | | neutral |
| Lys | polar, hydrophilic, | Trp | aromatic, hydrophobic, |
| | charged (+) | | neutral |
| Leu | aliphatic, hydrophobic, | Tyr | aromatic, polar, |
| | neutral | | hydrophobic |

Fragments of the IdeS polypeptide typically consist of no more than 100, 150, 200, 250, 300 or 350 contiguous amino acids of SEQ ID NOs: 1 or 2.

The amino acid sequence of any polypeptide, variant or fragment as described herein may be modified to include non-naturally occurring amino acids and/or to increase the stability of the compound. When the polypeptides are produced by synthetic means, such amino acids may be introduced during production. The polypeptides may also be modified following either synthetic or recombinant production. The polypeptides, variants or fragments described herein may be produced using D-amino acids. In such cases the amino acids will be linked in reverse sequence in the C to N orientation. This is conventional in the art for producing such polypeptides. A number of side chain modifications are known in the art and may be made to the side chains of the polypeptides, variants or fragments, subject to their retaining any further required activity or characteristic as may be specified herein.

It will also be understood that the polypeptides, variants or fragments may be chemically modified, e.g. post-translationally modified. For example, they may be glycosylated, phosphorylated or comprise modified amino acid residues.

The immunoglobulin containing sample used in the method of the invention may include immunoglobulin molecules such as IgM, IgA, IgD, and/or IgW, provided it includes at least one IgG molecule. Said IgG may be from any species, for example, human, monkey, rabbit, sheep or mouse, but is preferably human. Said IgG may be humanized or chimeric. The IgG may be Mouse IgG2a or IgG3. Preferably, the IgG is human or humanized IgG1, IgG2, IgG3 or IgG4.

Any suitable sample containing immunoglobulin molecules may be used in the method of the invention. For example, the sample may be an antibody clone, which is assessed to determine the ability of a particular clone to aggregate or remain in unaggregated form. The sample may be an antibody formulation, in order to assess the effect of a particular formulation on the aggregation properties of the antibody. Alternatively, the sample may be taken from a batch of synthetically produced immunoglobulins or IgG either before or after formulation for administration to a patient with a pharmaceutical carrier or diluent, in which the degree of aggregation is being assessed as part of the quality control for such a sample. The IgG antibody in the sample may be in the form of a monoclonal antibody such as a therapeutic monoclonal antibody; an antibody-drug conjugate or a bi-specific antibody.

The method of the invention may comprise the following steps:

(a) contacting a sample containing IgG immunoglobulin with the IdeS polypeptide;

(b) quantifying one or more IgG immunoglobulin cleavage fragments in the sample; and

(c) determining thereby the IgG immunoglobulin aggregation in the sample.

Step (a) may be performed under any conditions that permit the cleavage of IgG immunoglobulin molecules in the sample by the IdeS polypeptide. Suitable conditions are described in the Examples. Typically, any standard buffer is used at a pH of 5.0 to 8.0, such as 5.5 to 7.5, typically 6.0 to 7.5. Standard buffers include phosphate buffer saline (PBS), tris, ammonium bicarbonate, MES, HEPEs and sodium acetate. Typically, the sample is incubated with the first polypeptide for at least 20 minutes, at least 30 minutes, at least 40 minutes, at least 50 minutes, preferably at least 60 minutes. Incubation preferably takes place at room temperature, more preferably at approximately 20° C., 25° C., 30° C., 35° C., 40° C. or 45° C., and most preferably at approximately 37° C. Typically, the enzyme:antibody ratio is approximately 1:50 (w:v). Typically, a reducing agent is not used.

The quantification of cleavage may be identified by determining the quantity of Fc fragments, F(ab)₂ fragments or both in the sample using any suitable method. For example, Fc or F(ab)₂ fragments may be separated from the resulting mixture by affinity separation, size-exclusion chromatography (SEC), ion-exchange chromatography, gel filtration or dialysis. Typically, the mixture may be contacted with a suitable Fc binding agent. The mixture resulting from step (a) may be applied onto a human IgG Fc-binding resin and components other than Fc fragments, which do not bind to the resin (such as, for example, Fab fragments, the reducing agent and IdeS polypeptide), can be eluted off. Fc-binding agents such as human IgG Fc-binding resin are commercially available. Alternatively, a Fab binding agent is used to binding Fab fragments, and allow other components to be eluted off.

In a preferred aspect of the present invention, a high throughput method is used to separate and quantify the amount of cleavage products, such a Fc and/or F(ab′)2 fragments. Typically, the analysis involves a high throughput gel electrophoresis method, in which the components present in the sample are separated based on their size. Thus, F(ab′)2 and Fc fragments can be separated from each other and the amount of these fragments quantified.

Since the amount of cleavage products decreases with increased aggregation, then the amount of Fc or F(ab′)₂ fragments can be used to determine the amount of aggregation in the sample under investigation. In particular, the present inventors have determined that a linear regression model can be used to describe the reduced ability of IdeS to digest antibodies when aggregates are formed. In a method of the invention in which only Fc or F(ab′)2 fragments are quantified, then the concentration of antibody in the initial sample is also determined by any suitable technique or is known. In a preferred aspect of the present invention, both Fc and F(ab′)2 fragments are detected and the ratio between the peak areas for Fc and F(ab′)2 are used to determine the concentration of aggregates in the sample. In this aspect of the invention, it is not necessary to separately determine the critical antibody concentration.

Typically, the methods of the present invention are conducted on samples of antibodies taken, for example, from a production line for the production of that antibody such that the sample is tested for quality control purposes to confirm that the levels of aggregation of the antibodies remain at acceptable levels. Alternatively, the method can be used as part of the assessment to identify new antibodies for therapeutic, diagnostic or research use, or in formulating antibodies, to assess the ability of particular clones or formulations to be resistant to aggregation, and thus to identify antibodies or formulations with greater long term stability.

The following Examples illustrate the invention:

We applied forced aggregation to clinically approved monoclonal antibodies and exposed the resulting mixture of aggregates and free non-aggregated antibodies to IdeS digestion. Surprisingly we found that aggregated antibodies withstand IdeS digestion to a large extent. We found a direct relationship between aggregated IgG and the amount of IgG amenable to digestion by IdeS.

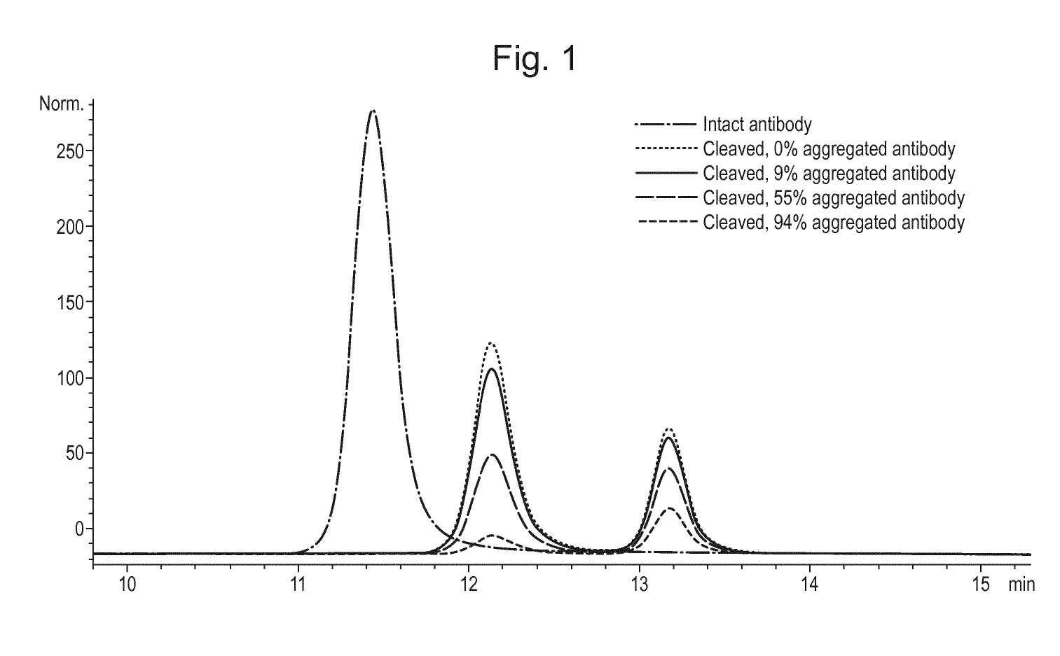

After SEC-HPLC analysis the peak for intact MabThera® was found after the retention time 11.4 minutes and the peaks for the F(ab′)2 and Fc segments after 12.1 and 13.2 minutes respectively, see FIG. 1.

In FIG. 1 the SEC-HPLC peak area for the different fragments in digested antibody solutions with different aggregate concentrations are plotted. Both the concentration of free F(ab′)₂ and free Fc decreases with increased aggregation, as was also confirmed from SDS-PAGE, indicating that IdeS cannot cleave large aggregates very well.

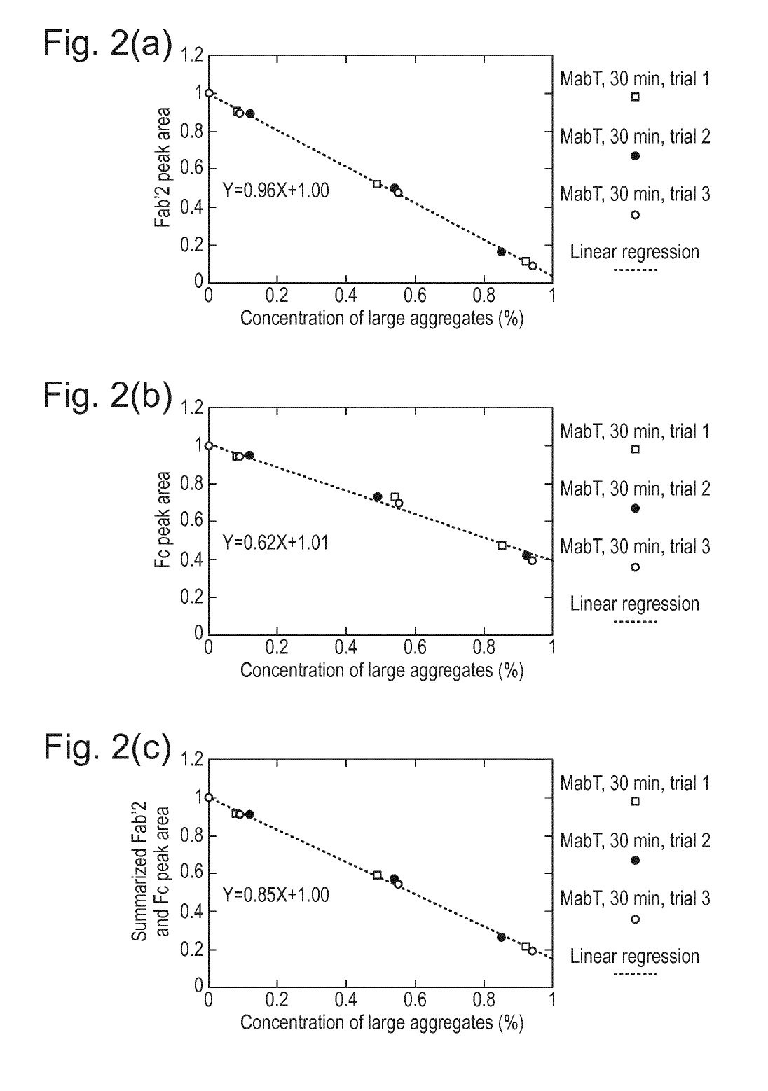

When fitting a linear regression model to either the decrease in F(ab′)₂ (FIG. 2a), the decrease in Fc (FIG. 2b) or the summarized product decrease (FIG. 2c) the linear regression model fits well giving coefficients of determination above 0.98. This indicates that a linear regression model can be used to describe the reduced ability of IdeS to digest antibodies when they form large aggregates.

High throughput Analysis of Degree of Aggregation using IdeS

Technologies as SDS-Page and capillary electrophoresis or combination thereof could be used to quantify the degree of aggregation using the discovered properties of IdeS activity above.

### Protocol Using Caliper's LabChip GXII System (Caliper Life Sciences)

### Antibody Digestion

55 μg crude antibody sample is added to wells on a 96 well microtiter plate and mixed with PBS buffer.
IdeS is added to the wells in an enzyme:antibody ratio of 1:50 (final antibody concentration in each well should be 1.1 mg/ml).
The plate is incubated in 37° C. for 30 min.

### HT Protein Express LabKit Analysis

7 μl of the kit denaturing solution is added to wells on a new microtiter plate, according to kit protocol.
2 μl of the content from each well is transferred to the new microtiter plate, according to kit protocol.
Then follow kit protocol (including dilution of sample with 35 ul water to 50 ng/μl).

Aggregation degree is then measured through calculating the ratio between F(ab′)₂ and Fc peak areas.

Since the detection limit of the device is 5 ng/μl the total antibody concentration must be 10× higher to detect free F(ab′)₂ and Fc concentrations down to 10% of total antibody concentration.

Since the analysis time of each sample with HT protein Express LabKit takes 41s (artikeln) analysis of a full 96 well plate should take slightly over 1 h. This means that the analysis time of the procedure as a whole should take under 2 h for 96 samples.

55 μg antibody is the detection limit for the HT protein Express LabKit if digestion volume is 50 μl.

The process could be further simplified to one microtiter plate and accomplished with lower antibody amounts if the kit protein preparation was optimized to this procedure. E.g. a higher denaturing solution concentration would lower the dilution of the sample and thereby the desired antibody amount to 49 μg if only 2 μl denaturing solution had to be added.

Protocol Using a cePRO 9600 96-Capillary Electrophoresis Instrument (CombiSep, Ames, Iowa, USA)

### Antibody Digestion

2.7 μg antibody sample is added to wells on a 96 well microtiter plate and mixed with Tris-HCl buffer.
Fabricator is added to the wells in an enzyme:antibody ratio of 1:50 (final antibody concentration in each well should be 54 μg/ml).
The plate is incubated in 37° C. for 30 min.
   cePRO 9600 96-Capillary Electrophoresis Analysis
2 μl 130 mM DTT solution is added to each well to a final concentration of 5 mM.
2 μl 2.7% SDS solution is added to each well to a final concentration of 0.5%.
Protein samples are denatured by heating the titer plate at 95° C. for 20 min before sample injection.
Follow protocol from Luo, S., J. Feng, H. Pang, 2004, High-throughput protein analysis by multiplexed sodium dodecyl sulfate capillary gel electrophoresis with UV absorption detection, Journal of Chromatography A, 1051 p. 131-134.

Aggregation degree is then measured through calculating the ratio between F(ab′)₂ and Fc peak areas.

Since the detection limit of the device is 5 ng/ul the total antibody concentration must be 10× higher to detect free F(ab′)₂ and Fc concentrations down to 10% of total antibody concentration.

Since 96 samples can be analyzed simultaneously within 30 min with the cePRO 9600 96-capillary electrophoresis instrument. This means that the analysis time of the procedure as a whole should take about 1 h for 96 samples.

2.7 μg antibody is the detection limit for the HT protein Express LabKit if digestion volume is 50 μl.

Sequence Listing

SEQ ID NO: 1
DSFSANQEIRYSEVTPYHVTSVWTKGVTPPANFTQGEDVFHAPYVANQGW
YDITKTFNGKDDLLCGAATAGNMLHWWFDQNKDQIKRYLEEHPEKQKINF
NGEQMFDVKEAIDTKNHQLDSKLFEYFKEKAFPYLSTKHLGVFPDHVIDM
FINGYRLSLTNHGPTPVKEGSKDPRGGIFDAVFTRGDQSKLLTSRHDFKE
KNLKEISDLIKKELTEGKALGLSHTYANVRINHVINLWGADFDSNGNLKA
IYVTDSDSNASIGMKKYFVGVNSAGKVAISAKEIKEDNIGAQVLGLFTLS
TGQDSWNQTN
SEQ ID NO: 2
MRKRCYSTSAAVLAAVTLFVLSVDRGVIADSFSANQEIRYSEVTPYHVTS
VWTKGVTPPANFTQGEDVFHAPYVANQGWYDITKTFNGKDDLLCGAATAG
NMLHWWFDQNKDQIKRYLEEHPEKQKINFNGEQMFDVKEAIDTKNHQLDS
KLFEYFKEKAFPYLSTKHLGVFPDHVIDMFINGYRLSLTNHGPTPVKEGS
KDPRGGIFDAVFTRGDQSKLLTSRHDFKEKNLKEISDLIKKELTEGKALG
LSHTYANVRINHVINLWGADFDSNGNLKAIYVTDSDSNASIGMKKYFVGV
NSAGKVAISAKEIKEDNIGAQVLGLFTLSTGQDSWNQTN

1. A method for quantifying the amount of IgG antibody aggregate in a sample of IgG immunoglobulin molecules comprising
(a) contacting the sample with a IdeS polypeptide under conditions which allow cleavage of an unaggregated IgG antibody,
(b) quantifying a cleavage product produced in step (a), and
(c) using the result of step (b) to determine the amount of antibody aggregation in the sample.

2. A method according to claim 1, wherein the IdeS polypeptide comprises or consists of the amino acid sequence of SEQ ID NO: 1, or a variant or fragment thereof.

3. A method according to claim 2, wherein the variant of SEQ ID NO:1 is an amino acid sequence having at least 80% identity to SEQ ID NO: 1 and wherein a fragment of SEQ ID NO: 1 comprises up to 300 contiguous amino acids of SEQ ID NO: 1.

4. A method according to claim 1, wherein said sample is a therapeutic antibody preparation.

5. A method according to claim 1, wherein said IgG molecule is a human or humanized IgG molecule.

6. A method according to claim 1, wherein the method comprises isolating Fc and/or F(ab′)₂ fragments from the sample, and quantifying the amount thereof.

7. A method according to claim 6, wherein said quantifying comprises a size separation technique such as gel electrophoresis to separate Fc fragments and F(ab′)₂ fragments from the mixture.

8. A method according to claim 1, wherein step (c) comprises determining the ratio of F(ab′)₂ to Fc fragments and using that ratio to determine the amount of aggregation in the sample.

9. A method according to claim 1, wherein the concentration of antibody in the sample is determined prior to contacting the sample with IdeS.


SEQUENCE LISTING (2 sequences)

SEQ ID NO: 1 - protein, 310 aa, Streptococcus pyogenes
DSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 2 - protein, 339 aa, Streptococcus pyogenes
MRKRCYSTSAAVLAAVTLFVLSVDRGVIADSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN